(19) United States
(12) Patent Application Publication
(10) Pub. No.: US 2018/0264121 A1
(43) Pub. Date: Sep. 20, 2018

(54) CANNABINOID-CONTAINING ORAL PHARMACEUTICAL COMPOSITION, METHOD FOR PREPARING AND USING SAME

(71) Applicants: PRATI-DONADUZZI & CIA LTDA., Toledo (BR); UNIVERSIDADE DE SÃO PAULO, São Paulo City (BR)

(72) Inventors: Luiz DONADUZZI, Toledo (BR); Carmen Maria DONADUZZI, Toledo (BR); Christian Gregory Burgos DE MENEZES, Toledo (BR); Liberato Brum JUNIOR, Toledo (BR); Volnei José Tondo FILHO, Toledo (BR); Patricia Moura ROSA, Toledo (BR); José Alexandre CRIPPA, Riberão Preto (BR); Jaime Eduardo Cecilio HALLAK, Riberão Preto (BR); Antonio Waldo ZUARDI, Ribeirão Preto (BR); Francisco Silveira GUIMARÃES, Ribeirão Preto (BR)

(73) Assignees: PRATI-DONADUZZI & CIA LTDA., Toledo (BR); UNIVERSIDADE DE SÃO PAULO, Sao Paulo City (BR)

(21) Appl. No.: 15/760,918

(22) PCT Filed: Sep. 16, 2016
(86) PCT No.: PCT/BR2016/050231

§ 371 Date: Mar. 16, 2018

(30) Foreign Application Priority Data
Sep. 18, 2015 (BR) BR 102015024165-8

(51) Int. Cl. A61K 47/44; A61K 9/00; A61K 31/05; A61K 47/10

(52) U.S. Cl. CPC A61K 47/44; A61K 47/10; A61K 31/05; A61K 9/0053

(57) ABSTRACT

The present invention describes an oral pharmaceutical composition comprising cannabinoid(s), an oily liquid solvent and a co-solvent, as well as a process for preparing and the same for the treatment of neurological disorders, especially refractory epilepsy. The analytical methods used to ensure identity, quality and purity of Active Pharmaceutical Ingredient and formulation produced were validated and have defined specifications.

### FIELD OF THE INVENTION

The present invention describes an oral pharmaceutical composition comprising cannabinoid(s) in an oily liquid carrier, as well as a process for its preparation.

The present invention is useful in the treatment of neurological disorders, particularly refractory epilepsy.

The present invention describes analytical method and specifications for quality control of Active Pharmaceutical Ingredient (API) and oral pharmaceutical formulation containing cannabidiol.

The present invention describes a study for safety and efficacy evaluation of oral formulation containing cannabidiol.

### BACKGROUND OF THE INVENTION

The plants of the genus Cannabis (Cannabis sativa, Cannabis ruderalis and Cannabis indica) have various substances of interest in science and medicine, so-called cannabinoids, for example: Δ9-tetrahydrocannabinol (D9-THC), Δ8-tetrahydrocannabinol (D8-THC), tetrahydrocannabinol acid (THC-A), tetrahydrocannabivarin (THCV), tetrahydrocannabinoline (THC-A), tetrahydrocannabinoline (THCV-A), tetrahydrocannabinoline acid (THCV-A), cannabidiol (CBD), cannabidiol acid (CBD-A), cannabichromene (CBC), cannabidivarine (CBDV), cannabidivarine acid (CBDV-A), cannabigerol (CBG), cannabigerol-acid (CBG-A), cannabigerovarine (CBGV), cannabinol (CBN), cannabinovarin (CBNV), among others. Scientific research was able to identify a total of 85 cannabinoids isolated from Cannabis plants, and their use has been intensely investigated for the treatment of epilepsy, nausea, vomiting, loss of appetite, pain and inflammatory diseases, among other pathologies.

Epilepsy is the most common neurological disorder of all, being characterized by recurrent seizures, the result of excessive electrical discharges from neurons. It affects around 50 million people worldwide and approximately 40% of all patients have seizures that are not fully controlled with drugs.

The effects from this situation affect not only the health system, but also the patients and their families, since this is a disease in which individuals lose their independence, compromising their socialization and their psychological development. In addition, cognitive performance may be affected by epilepsy, as well as by current therapy.

In this context, the use of other cannabinoids has been shown to be a promising alternative for patients not only for refractory epilepsy, but also for other neurological disorders.

The scientific literature contains numerous studies on the efficacy of cannabinoids in the treatment of epilepsy. Various patent applications, for example BR 11 2012 000076 4, WO 2012/093255, BR 11 2012024480 9, WO 2009/087351 and WO 2007/148094 express such use and claim methods of treatment and medicaments.

However, such documents express such drugs very generically without detailing the aspects of the technique involved and difficulties a skilled person would encounter in developing such formulation. In addition, most of the experimental data are carried out in mice and mice, from intra-peritoneal injections, an impracticable approach in humans.

The present invention goes beyond these general teachings, describing and proving the feasibility of an oral liquid formulation constituted by cannabinoid(s).

WO 2009/020666 discloses an oral liquid composition of cannabinoids, wherein the solvent is aqueous, and has a more efficient in vivo absorption profile than commercial gelatin capsules.

U.S. Pat. No. 7,025,992 describes a cannabinoid formulation as powder, which when hydrated, has its absorption facilitated by means of an emulsifier.

WO 2015/068052 describes liposomes comprising cannabinoids and terpenes.

The present invention differs from the others in that it is an oily media soluble formulation which promotes the bioavailability of the active ingredient when compared to the powder pharmaceutical form.

Moreover, it is important to emphasize that all the documents of the technique point to the gelatinous capsules orally as an inefficient form for administration of the cannabinoids, with aqueous solvents being used as the alternative. The present invention challenges this dogma, and shows that it is possible to provide an oily formulation with an increased bioavailability. Thus, the teachings of the present invention should be considered novel and inventive.

### SUMMARY OF THE INVENTION

In a first aspect, the present invention provides an oral liquid composition for improving the bioavailability, stability and organoleptic properties of cannabinoid.

It is an object of the present invention to provide an oral liquid composition comprising cannabinoids. In particular, such composition comprises an oily solvent.

In a preferred embodiment, the oily solvent optionally comprises a co-solvent.

An additional object of the present invention is a process of preparing an oral liquid composition comprising the steps of:

a. Dissolution of cannabinoid:

i. in the oily solvent; or

ii. in the co-solvent, followed by addition and homogenization to the oily solvent;

b. addition of the excipients and homogenization.

It is a further object of the present invention the use the oral liquid composition in the treatment of neurological disorders such as refractory epilepsy, epilepsy, Parkinson's disease, schizophrenia, sleep disorders, post-traumatic disorder, chronic pain relief, and autism.

### DETAILED DESCRIPTION OF THE INVENTION

The examples shown herein are only intended to exemplify some of the numerous embodiments of the invention, and therefore, should not be considered to limit the teachings herein attached.

Liquid Oral Composition Comprising Cannabinoids

For the purposes of the present invention, the term “cannabinoids” means substances capable of activating the cannabinoid receptors present in the cells. They can be chosen from endocannabinoids, phytocannabinoids and synthetic cannabinoids.

Endocannabinoids are man-made substances capable of active cannabinoid receptors. Non-limiting examples of endocannabinoids include anandamide (AEA), 2-arachidonoylglycerol (2-AG), 2-arachidonyl glyceryl ether, N-arachidonoyl dopamine (NADA), virodhamine (OAE) and comb6]. Phytocannabinoids are naturally occurring cannabinoids that can be found in plants of the Cannabis genus. Phytocannabinoids may be present in the form of extract, isolated or synthetically reproduced compounds. Non-limiting examples of phytocannabinoids include Δ9-tetrahydrocannabinol (D9-THC), Δ8-tetrahydrocannabinol (D8-THC), tetrahydrocannabinol acid (THC-A), tetrahydrocannabivarin (THCV), tetrahydrocannabivarin acid (THCV-A) cannabidiol (CBD), cannabidiol acid (CBD-A), cannabichromene (CBC), cannabidivarine (CBDV), cannabidivarine acid (CBDV-A), cannabigerol (CBG), cannabigerol acid (CBG-A), cannabigerovarine (CBGV), cannabinol (CBN), cannabinovarin (CBNV), and combinations thereof.

Synthetic cannabinoids are compounds capable of interacting with cannabinoid receptors and are not found either endogenously or in plants. Non-limiting examples of synthetic cannabinoids include dronabinol, nabilone, rimonabant, and combinations thereof.

Within the scope of the present invention the pharmaceutically acceptable salts and acids of the above-mentioned compounds are also contemplated.

Preferably, among the cannabinoids mentioned, it is highlighted the cannabidiol, its pharmaceutically acceptable acid, or combinations thereof.

The concentration of the cannabinoid in the composition ranges from 5 to 500 mg/ml, more preferably from 20 to 250 mg/ml.

The oily solvent of the present invention is an oily selected from the group consisting of grape seed oil, sesame oil, corn oil, soybean oil, olive oil, sunflower oil, canola oil, walnut oil, linseed oil, avocado oil, peppermint oil, peanut oil, hydrogenated castor oil, coconut oil, acai oil, andiroba oil, babassu oil, buriti oil, Brazil nuts oil, copaiba oil, passion fruit oil, pracaxi oil, patawa oil, triglycerides, primrose oil, safflower oil, almond oil, borage oil, pomegranate seed oil, sea buckthorn oil, garlic oil, krill oil, cod liver oil, palm oil, macadamia oil, musk rose oil, cotton oil, and combinations thereof.

Preferably the oil used is the corn oil.

The composition optionally comprises a co-solvent to aid in the solubilization of the cannabinoid in the oily solvent.

Non-limiting examples of co-solvents include propylene glycol, glycerol, polyethylene glycol, ethanol, and combinations thereof.

Excipients used in the composition of the present invention are the excipients commonly found in the state of art and known to those skilled in the art. Non-limiting examples include antioxidants, sweeteners, flavorings, preservatives, and combinations thereof.

The antioxidant may be selected from acetylcysteine tocopherols, α-tocopherol, d-α-tocopherol, DL-α-tocopherol, ascorbic palmitate, butylhydroxyanisole (BHA), butylhydroxytoluene (BHT), lecithin, cysteine, cysteine hydrochloride, propyl gallate, ascorbic acid, iso-ascorbic acid, thioglycerol, citric acid, tartaric acid, EDTA and its salts, hydroxyquinoline sulfate, phosphoric acid, sodium metabisulphite and sodium citrate, t-butylhydroquinone (TBHQ), and combinations thereof.

The sweetening agents may be selected from sucralose, saccharin, sodium saccharin, sodium cyclamate, sorbitol, xylitol, sucrose, glycerol, neohesperidine, aspartame, and combinations thereof. The amount of sweetening agent used may be in the range of about 0.05 to about 3% by weight of the composition.

The flavoring agents may be selected from peppermint, mint, lemon, strawberry, mango, pear, peach, raspberry, plum, pineapple aroma and the like. The amount of flavoring used ranges from about 0.05 to about 5% by weight of the composition.

Preservatives may be selected from sodium benzoate, potassium sorbate, benzyl alcohol, parabens esters (p-hydroxybenzoic acids) such as methylparaben, ethylparaben, propylparaben, butylparaben, and the like, and combinations thereof. The amount of preservatives used ranges from about 0.01 to about 2% by weight.

The oral compositions of the present invention have cannabinoid concentrations within the specifications set forth in the analytical methodology object of this patent, remaining stable for long periods of time, including the storage period and validity, without appreciable occurrence of degradations such as Δ9-tetrahydrocannabinol and cannabinol, the main psychoactive compounds.

The present invention is further an acceptable alternative for patients, for example, children and the elderly, who cannot or do have difficulties in taking tablets or because of their clinical condition at the start of the treatment, make it impossible to swallow solid pharmaceutical formulas.

Preparing Process

The preparation process of the composition of the present invention comprises the steps of:

a. Dissolution of cannabinoid:

i. in the oily solvent; or

ii. in the co-solvent, followed by addition and homogenization to the oily solvent;

b. addition of the excipients and homogenization.

Dissolution of the cannabinoid in the oily solvent occurs at temperatures between 20° C. and 100° C., preferably between 30° C. and 80° C. and with stirring. When a co-solvent is used, the cannabinoid is first dissolved in the co-solvent, under the same conditions described above, and then such solution is added to the oily solvent.

The excipients are added after cooling of the solution, and kept under stirring until complete solubilization.

The entire process of manufacturing and packaging the product may be carried out under an inert gas environment such as nitrogen, argon, helium or the like.

The productive process was validated considering critical points and control steps according to the table below:

| STEP | CRITICAL POINT | CONTROL |
|---|---|---|
| PREPARATION | TIME, STIRRING | EVALUATE AT LEAST 3 |
| | SPEED, | PRODUCTIVE BATCHES |
| | SOLUBILITY AND | FOLLOW AND REGISTER |
| | TEMPERATURE. | PARAMETERS IN THE ORDER OF |
| | | MANUFACTURE |
| | | AT THE END OF THE |
| | | PRODUCTION PROCESS, |
| | | SAMPLES ARE REMOVED FOR |
| | | THE FOLLOWING ANALYSIS: |
| | | DOSING, RELATED |
| | | SUBSTANCES AND |
| | | MICROBIOLOGICAL ANALYSIS |
| PROCESS OF | NOT APPLICABLE | EVALUATE AT LEAST 3 |
| TRANSFER OF | | BATCHES |
| THE CASTER TO | | FOLLOW AND REGISTER |
| THE STOCKING | | PARAMETERS IN THE ORDER OF |
| TANK | | MANUFACTURE |
| PRIMARY | PACKAGING | EVALUATE THE MINIMUM 3 |
| PACKAGING | SPEED | PILOT BATCHES |
| | | FOLLOW AND REGISTER |
| | | PARAMETERS IN THE ORDER OF |
| | | MANUFACTURE |
| | | DURING PRODUCTION |
| | | PROCESS THE SAMPLES ARE |
| | | REMOVED FOR PACKAGING |
| | | VOLUME, BOTTLE OPENING AND |
| | | BOTTLE LEAKAGE ANALYSIS. |

Use of the Composition

The present invention is useful in the treatment of neurological disorders, such as refractory epilepsy, epilepsy, Parkinson's disease, schizophrenia, sleep disorders, post-traumatic disorder, anxiety and chronic pain relief.

The effective dose of cannabinoid ranges from 1 to 50 mg/kg/day of body weight, preferably from 2.5 to 25 mg/kg/day, and may be in single dose or divided throughout the day.

Safety and Efficacy Assessment for Refractory Epilepsy Indication

After a 4-week baseline period to complete a seizures diary, research participants who meet the inclusion criteria will be randomized to enter the 17-week treatment period. The treatment period will consist of two phases: Titration and Maintenance or treatment per se. In the Titration Phase, the physicians responsible for the follow-up of the research participants will be instructed to blind prescribe the developed formulation object of this patent or placebo with adjuvant therapy to the medications that the patients have been using, starting with a volume in ml of the research solution attributed to the research participant, which would correspond to a dose of 5 mg/kg/day. The solution researched should be increased in the same volume (corresponding to a dose of 5 mg/kg/day of CBD) every 7 days, if there were seizures during the previous period up to the dosage of 25 mg/kg/day or occurrence of clinically significant adverse event. In the Maintenance Phase, it is intended to maintain the highest dose obtained in the stable titration period until the end of the study.

The need for dose reduction will be allowed and evaluated if the research participant shows an adverse event related to the dose of the experimental drug. Dose escalation is allowed up to the maximum dose of 25 mg/kg/day, if after determination of the “ideal” maintenance dose the patient's seizures have returned.

Inclusion criteria for patients: An approved informed consent form (ICF) by the Ethics Committee (EC) must have been signed and dated by the legal representative; Research subjects of both genders within the age range of 6 months to 18 years; Diagnosis of refractory epilepsy characterized according to International League Against Epilepsy (ILAE) classification; Research subjects who show at least 4 epileptic seizures during the 4-week Baseline Period, with an interval between seizures that does not exceed 21 days; In treatment with up to 03 concomitant AEDs at stable doses at least 1 month before the initial visit and with the expectation of remaining stable during the treatment period. The vagus nerve stimulator (VNS) is considered as an AED; Legal representative considered capable of complying with the protocol (for example, able to understand and fill in the diaries), visiting schedule or medication intake, according to the investigator's opinion; Presence of encephalic neuroimaging (Magnetic Resonance Image (MRI) or Computed Tomography (CT)) performed within the last 5 years; Without significant co-morbidities, the physician's judgment, according to the other criteria adopted in this Protocol and evaluations to which he/she was subjected: clinical history, pressure, pulse and temperature measurement, physical examination, ECG, EEG and laboratory tests and Research subjects with potential to be pregnant can be included in the study provided they are in sexual abstinence or using contraceptive method considered effective.

Exclusion Criteria: Occurrence of only simple partial seizures (preserved consciousness) without motor symptomatology; History or presence of pseudo-seizures; History of attempted suicide; History of major depression; Pregnant research participant; Research subject in use of drug abuse; Hypertensive research subject; Research subject with severe dysphagia who is not using a gastric or naso-gastric tube; Research subject in drug use research that may significantly influence the metabolism of CBD, except for AED if stable for at least 1 month prior to the initial visit; Presence of any clinical or neuroimaging signal suggesting brain disorder, brain tumor, metabolic or neurodegenerative disease with quick progression; Presence of known acute and clinically significant illness at the examining physician's criteria, such as renal, hepatic, urinary, intestinal, respiratory infections; Presence of known and clinically significant chronic disease at the examining physician's criteria that may compromise study participation or pose a safety risk to the research subject; History of liver, renal, pulmonary, gastrointestinal, hematological, heart or psychiatric disease that, in the clinical criteria, could compromise the health of the research subject and/or his/her participation in the study; Hypotension or hypertension of any etiology requiring pharmacological treatment; Surgical history that may compromise the health of the research subject and/or his/her participation in the study; Research subject in regular or intermittent use of marijuana in the last 60 days prior to the initial visit; Research subject in regular or intermittent CBD-based treatment in the last 60 days prior to the initial visit; History of allergy or idiosyncratic reactions to Cannabis Sativa derivatives or components of the pharmaceutical formulation; ECG abnormalities considered clinically significant according to the investigator; Subjects who have participated in another clinical study less than 3 months before the date of the Initial Visit; Donation or loss of 450 ml or more of blood within 90 days (three months) prior to the date of the Initial Visit; Liver function impairment: TGO, TGP, alkaline phosphatase and yGT values more than 3 times above the upper limit of the reference range. A result of yGT that exceeds 3 times the upper limit only may be acceptable if it is attributable to hepatic enzyme induction caused by concomitant treatment with AED and the other liver enzymes are below 3 times the upper limit of the reference range and Research subject with clinically significant deviations from the reference range values for the following laboratory parameters: creatinine clearance <50 ml/min, platelets <100,000/μL, or neutrophils <1800/μL.

Validated Analytical Methodology and Acceptable Limits to Ensure Identity, Quality and Purity of the API.

| EVALUATED PARAMETER | SPECIFICATIONS | |
|---|---|---|
| DESCRIPTION | CRYSTALLINE OR NEAR WHITE POWDER | |
| SOLUBILITY | INSOLUBLE IN PURIFIED WATER, SOLUBLE | |
| | IN ETHANOL 96%, CHLOROFORM AND | |
| | SLIGHTLY SOLUABLE IN HEXANE | |
| IDENTIFICATION A | SAMPLE SPECTRUM SAMPLE SHOWS | |
| | MAXIMUM ABSORPTION ONLY IN THE SAME | |
| | WAVELENGTHS AND WITH THE SAME | |
| | RELATIVE INTENSITIES OF THOSE | |
| | OBSERVED IN THE STANDARD SPECTRUM | |
| IDENTIFICATION B | THE MAIN PEAK RETENTION TIME OBTAINED | |
| | ON THE SAMPLE SOLUTION | |
| | CHROMATOGRAM CORRESPONDS TO THE | |
| | PEAK RETENTION TIME OF THE STANDARD | |
| | SOLUTION (1). | |
| FUSION POINT | 63 TO 69° C. | |
| SPECIFIC ROTATION | SPECIFIC OPTICAL ROTATION MUST 120° | |
| | TO −130° IN RELATION TO THE ANHYDROUS | |
| | SUBSTANCE | |
| WATER | ≤0.5% | |
| WATER ACTIVITY | ≤0.600% | |
| HEAVY METALS | ANY COLOR DEVELOPED BY THE TEST | |
| | SOLUTION IS NOT MORE INTENSE THAT IT IS | |
| | DEVELOPED BY THE CONTROL SOLUTION | |
| SULPHATED ASH (2) | ≤0.1% | |
| RELATED SUBSTANCES | CANNABINOL | ≤0.14% |
| AND DEGRADATION | DELTA 9 TETRA- | ≤0.14% |
| PRODUCTS (1) | HYDROCANNABINOL | |
| | INESPECIFIC INDIVIDUAL | ≤0.14% |
| | IMPURITIES | |
| | TOTAL IMPURITIES | ≤0.42% |
| DOSING (1) | 98-102% (1) | |
| RESIDUAL SOLVENTS (1) | ACETONE | ≤5000 μg · g⁻¹ |
| | HEPTANE | ≤5000 μg · g⁻¹ |
| | BENZENE | ≤2 μg · g⁻¹ |

Validation of analytical methodology for API content and dosing.

| EVALUATED | |
|---|---|
| PARAMETER | SPECIFICATIONS |
| SPECIFICITY AND | PROOF THAT THE PEAK QUANTIFIED IN THE |
| SELECTIVITY | CHROMATOGRAM OF THE SOLUTION TEST IS |
| | REALLY THE ANALYTE OF INTEREST (CANABIDIOL) |
| | THROUGH THE COMPARISON OF THE RETENTION |
| | TIME OF THIS PEAK WITH THE RETENTION TIME OF |
| | CANABIDIOL IN THE STANDARD SOLUTION. |
| | PROOF THAT NO SUBSTANCE CO-ELUATES |
| | (RESOLUTION EQUAL TO OR MORE THAN 1.5) WITH |
| | THE CANABIDIOL PEAK IN THE TEST SOLUTION |
| | THROUGH THE RAW MATERIAL SAMPLES ANALYSIS |
| | SUBMITTED TO FORCED DEGRADATION AND |
| | POSTERIOR VERIFICATION OF CANABIDIOL PEAK |
| | PURITY IN THESE SOLUTIONS. THE RESULTS OF |
| | THE PEAK PURITY ARE EXPRESSED BY |
| | COMPARING THE PURITY ANGLE WITH THE PURITY |
| | THRESHOLD WHEN PURITY ANGLE VALUE IS LESS |
| | THAN THRESHOLD PURITY VALUE, PEAK IS PURE |
| | AND, ON CONTRARY, PEAK IS IMPURE. |
| LINEARITY | CORRELATION COEFFICIENT (R) MUST BE EQUAL |
| | OR GREATER THAN 0.99. |
| | DO NOT PRESENT TREND IN THE DISTRIBUTION |
| | OF RESIDUES. |
| | RELATIVE STANDARD DEVIATION (RSD) BETWEEN |
| | THE 3 SAMPLES OF EACH CONCENTRATION LEVEL, |
| | AND IT SHOULD NOT EXCEED 2.0% FOR |
| | CONCENTRATION 100 μg/mL TO <1000 μg/mL. |
| INTERVAL | THE INTERVAL OF THE ANALYTICAL |
| | METHODOLOGY WILL BE ESTABLISHED BY THE |
| | CONFIRMATION THAT THE METHOD PROVIDES |
| | PRECISION, ACCURACY AND LINEARITY SUITABLE |
| | WITHIN 80% TO 120% RANGE OF THE NOMINAL |
| | CONCENTRATION OF THE SAMPLE IN THE DOSING |
| | ANALYTICAL METHODOLOGY (200.0 μg/mL). |
| ACCURACY | THE RECOVERY AVERAGE IN THREE LEVELS (80%, |
| | 100% AND 120% OF THE NOMINAL |
| | CONCENTRATION OF CANABIDIOL IN THE SAMPLE) |
| | MUST BE BETWEEN 98% AND 102% |
| REPEATABILITY | RELATIVE STANDARD DEVIATION (RSD) BETWEEN |
| 1^{ST} AND 2^{ND} DAY | SIX DETERMINATIONS SHOULD BE MINOR OR |
| ACCURACY | EQUAL 2.0%. |
| INTERMEDIARY | RELATIVE MEAN DIFFERENCE BETWEEN FIRST |
| ACCURACY | REPETIBILITY MEANS AND SECOND MUST BE LESS |
| | OR EQUAL 2.0% |
| ROBUSTNESS | METHOD WILL BE CONSIDERED ROBUST FOR |
| | SUCH VARIATIONS WHEN THE RESOLUTION |
| | BETWEEN THE CANABIDIOL PEAK AND ANY OTHER |
| | PEAK IS NOT LESS THAN 1.5 AND THE TEST |
| | SOLUTION CONTENT WILL NOT VARY MORE THAN |
| | 2.0% OF THE CONTENT OBTAINED WITHOUT |
| | CHANGE IN ANALYTICAL METHODOLOGY. |

Validation of analytical methodology for related substances and degradation product of API

| EVALUATED | |
|---|---|
| PARAMETER | SPECIFICATIONS |
| SPECIFICITY | PROOF THAT THE PEAK QUANTIFIED IN THE |
| | CHROMATOGRAM OF THE TEST SOLUTION IS |
| | REALLY THE ANALYTE OF INTEREST (CANABIDIOL) |
| | THROUGH THE COMPARISON OF THE RETENTION |
| | TIME OF THIS PEAK WITH THE RETENTION TIME OF |
| | CANABIDIOL IN THE STANDARD SOLUTION. |
| | PROOF THAT ANY DEGRADATION PRODUCT CO- |
| | ELUATES (RESOLUTION EQUAL TO OR MORE THAN |
| | 1.5) WITH THE CANABIDIOL PEAK, Δ9-THC AND CBN |
| | IN THE TEST SOLUTION THROUGH THE SAMPLES |
| | ANALYSIS SUBMITTED TO FORCED DEGRADATION IN |
| | ACID, BASE, OXIDATIVE, THERMAL, PHOTOLYTIC |
| | MEDIUM, HUMIDITY AND METAL ION AND POSTERIOR |
| | VERIFICATION OF CANABIDIOL PEAK PURITY IN |
| | THESE SOLUTIONS AND POSTERIOR VERIFICATION |
| | OF CANABIDIOL PEAK PURITY, Δ9-THC AND CBN (IF |
| | FORMED) IN THESE SOLUTIONS. THE RESULTS OF |
| | THE PEAK PURITY ARE EXPRESSED BY COMPARING |
| | THE PURITY ANGLE WITH THE PURITY THRESHOLD |
| | WHEN PURITY ANGLE VALUE IS LESS THAN |
| | THRESHOLD PURITY VALUE, PEAK IS PURE AND, ON |
| | CONTRARY, PEAK IS IMPURE. |
| LINEARITY | CORRELATION COEFFICIENT (R) MUST BE EQUAL |
| | OR GREATER THAN 0.99. |
| | DO NOT PRESENT TREND IN THE DISTRIBUTION OF |
| | RESIDUES. |
| | RELATIVE STANDARD DEVIATION (RSD) BETWEEN |
| | THE 3 SAMPLES OF EACH CONCENTRATION LEVEL, |
| | AND IT SHOULD NOT EXCEED 11.0% FOR |
| | CONCENTRATION <1 μg/mL TO <10 μg/mL AND |
| | SHOULD NOT EXCEED 7.3% FOR CONCENTRATION ≥10 μg/mL |
| | TO 100 μg/mL. |
| RESPONSE | RELATIVE RESPONSE FACTOR WILL BE OBTAINED |
| FACTOR | THROUGH THE RATION BETWEEN THE ANGULAR |
| | COEFFICIENTS FROM IMPURITIES AND ACTIVE, |
| | OBTAINED FROM THE LINEARITY PARAMETER. |
| LIMIT OF | THIS PARAMETER WAS DETERMINED FROM THE |
| QUANTIFICATION | RESULTS OF THE LINEARITY PARAMETER. |
| INTERVAL | THIS PARAMETER IS ESTABLISHED BY THE |
| | CONFIRMATION THAT THE METHOD PROVIDES |
| | PRECISION, ACCURACY AND LINEARITY SUITABLE |
| | WHEN APPLIED TO SAMPLES CONTAINING |
| | SUBSTANCE QUANTITIES WITHIN THE SPECIFIED |
| | INTERVAL. |
| ACCURACY | DIFFERENCE BETWEEN THE ADDED |
| | (THEORETICAL) AND THE RECOVERED |
| | (EXPERIMENTAL) AMOUNTS SHOULD BE BETWEEN |
| | 80% AND 110% FOR IMPURITY CONCENTRATION <1 μg/mL; |
| | BETWEEN 80% AND 110% FOR IMPURITY |
| | CONCENTRATION ≥1 μg/mL AND BETWEEN 99% AND |
| | 107% FOR IMPURITY CONCENTRATION ≥10 μg/mL TO |
| | <100 μg/mL. |
| | RSD BETWEEN 3 SAMPLES OF EACH |
| | CONCENTRATION LEVEL (0.05%, 0.14%, 1.00% AND |
| | 2.00% OF THE NOMINAL CONCENTRATION OF |
| | CANABIDIOL OF 4000,00 μg/mL) AND IT SHOULD NOT |
| | EXCEED 15% FOR IMPURITY CONCENTRATION <1 μg/mL; |
| | 11% FOR IMPURITY CONCENTRATION ≥1 μg/mL |
| | TO <10 μg/mL AND 7.3% FOR IMPURITY |
| | CONCENTRATION ≥10 μg/mL TO <100 μg/mL. |
| REPEATABILITY | REPETIBILITY RESULT EVALUATION WILL BE |
| | PERFORMED BY ASSESSING THE RELATIVE |
| | STANDARD DEVIATION OF EACH LEVEL, THIS |
| | SHOULD NOT EXCEED 15% FOR IMPURITY |
| | CONCENTRATION <1 μg/mL; 11% FOR IMPURITY |
| | CONCENTRATION ≥1 μg/mL TO <10 μg/mL AND 7.3% |
| | FOR IMPURITY CONCENTRATION ≥10 μg/mL TO <100 μg/mL. |
| INTERMEDIARY | INTERMEDIATE ACCURACY WILL BE ASSESSED |
| ACCURACY | THROUGH THE VARIATION BETWEEN THE MEANS OF |
| | THE CONTENTS OBTAINED AT EACH |
| | CONCENTRATION LEVEL ON THE FIRST AND ON THE |
| | SECOND DAY OF ANALYSIS, WHICH DIFFERENCE |
| | SHOULD BE UPPER 15% FOR IMPURITY |
| | CONCENTRATION <1 μg/mL; 11% FOR IMPURITY |
| | CONCENTRATION ≥1 μg/mL TO <10 μg/mL AND 7.3% |
| | FOR IMPURITY CONCENTRATION ≥10 μg/mL TO <100 μg/mL. |
| ROBUSTNESS | RESOLUTION BETWEEN THE PEAKS OF |
| | DEGRADATION AND THE CANABIDIOL PEAK ON |
| | EACH CHROMATOGRAM SHOULD NOT BE LESS |
| | THAN 1.5 AND VARIABLES BETWEEN THE CONTENT |
| | OF THE ACCURACY SOLUTION AND THE CONTENT |
| | OBTAINED WITH THE METHOD WITHOUT CHANGES |
| | SHOULD NOT BE UPPER 15.0% WITH RECOVERY OF |
| | 80 TO 110% FOR IMPURITY CONCENTRATION <1 μg/mL, |
| | SHOULD NOT BE UPPER 11.0% WITH |
| | RECOVERY; BETWEEN 80 TO 110% FOR IMPURITY |
| | CONCENTRATION ≥1 μg/mL TO <10 μg/mL, SHOULD |
| | NOT BE UPPER 7.3% WITH RECOVERY BETWEEN 90 |
| | TO 107% FOR IMPURITY CONCENTRATION ≥10 μg/mL |
| | TO <100 μg/mL. |

Validated Analytical Methodology and Acceptable Limits to Ensure identity, quality and purity of the formulation object of this patent.

| TEST | SPECIFICATIONS | |
|---|---|---|
| DESCRIPTION | OILY LIQUID, BRIGHT AND GOLDEN | |
| | YELLOW COLOR | |
| IDENTIFICATION | MAIN PEAK RETENTION TIME | |
| | OBTAINED IN THE TEST | |
| | SOLUTION CHROMATOGRAM | |
| | CORRESPONDS TO THE PEAK | |
| | RETENTION TIME OBTAINED | |
| | IN THE STANDARD SOLUTION | |
| | CHROMATOGRAM | |
| VOLUME | AVERAGE VOLUME | ≥30 mL |
| DETERMINAÇÃO | LOWER LIMIT | ≥95.0% |
| RELATED SUBSTANCES | CANNABINOL | 0.20% |
| | DELTA 9 TETRA- | 0.20% |
| | HYDROCANNABINOL | |
| | INESPECIFIC INDIVIDUAL | 0.20% |
| | IMPURITIES | |
| | TOTAL IMPURITIES | 0.20% |
| DOSING | 180 mg/mL-220 mg/mL | |

| | |
|---|---|
| BACTERIA TOTAL COUNT | ≤100 UFC/mL |
| MOULDS AND YEAST | ≤10 UFC/mL |
| TOTAL COUNT | |
| ESCHERICHIA COLI | ABSENT |

Validation of Analytical Methodology for Formulation Content and Dosing Object of this Patent.

| EVALUATED | |
|---|---|
| PARAMETER | SPECIFICATIONS |
| SPECIFICITY AND | PROOF THAT THE PEAK QUANTIFIED IN THE |
| SELECTIVITY | CHROMATOGRAM OF THE TEST SOLUTION IS |
| | REALLY THE ANALYTE OF INTEREST (CANABIDIOL) |
| | BY COMPARING OF THE RETENTION TIME OF THIS |
| | PEAK WITH THE RETENTION TIME OF CANABIDIOL |
| | IN THE STANDARD SOLUTION. |
| | PROOF THAT NO SUBSTANCE CO-ELUATES |
| | (RESOLUTION EQUAL TO OR MORE THAN 1.5) WITH |
| | THE CANABIDIOL PEAK IN THE TEST SOLUTION |
| | THROUGH THE RAW MATERIAL SAMPLES ANALYSIS |
| | SUBMITTED TO FORCED DEGRADATION AND |
| | POSTERIOR VERIFICATION OF CANABIDIOL PEAK |
| | PURITY IN THESE SOLUTIONS. THE RESULTS OF |
| | THE PEAK PURITY ARE EXPRESSED BY |
| | COMPARING THE PURITY ANGLE WITH THE PURITY |
| | THRESHOLD WHEN PURITY ANGLE VALUE IS LESS |
| | THAN THRESHOLD PURITY VALUE, PEAK IS PURE |
| | AND, ON CONTRARY, PEAK IS IMPURE. |
| LINEARITY | CORRELATION COEFFICIENT (R) MUST BE EQUAL |
| | OR GREATER THAN 0.99. |
| | DO NOT PRESENT TREND IN THE DISTRIBUTION |
| | OF RESIDUES. |
| | RELATIVE STANDARD DEVIATION (RSD) BETWEEN |
| | THE 3 SAMPLES OF EACH CONCENTRATION LEVEL, |
| | AND IT SHOULD NOT EXCEED 2.0% FOR |
| | CONCENTRATION 100 μg/mL TO <1000 μg/mL. |
| INTERVAL | THE INTERVAL OF THE ANALYTICAL |
| | METHODOLOGY WILL BE ESTABLISHED BY THE |
| | CONFIRMATION THAT THE METHOD PROVIDES |
| | PRECISION, ACCURACY AND LINEARITY SUITABLE |
| | WITHIN 80% TO 120% RANGE OF THE NOMINAL |
| | CONCENTRATION OF THE SAMPLE IN THE DOSING |
| | ANALYTICAL METHODOLOGY (200.0 μg/mL). |
| ACCURACY | THE RECOVERY AVERAGE IN THREE LEVELS (80%, |
| | 100% AND 120% OF THE NOMINAL |
| | CONCENTRATION OF CANABIDIOL IN THE SAMPLE) |
| | MUST BE BETWEEN 98% AND 102% |
| REPEATABILITY | RELATIVE STANDARD DEVIATION (RSD) BETWEEN |
| 1^{ST} AND 2^{ND} DAY | SIX DETERMINATIONS SHOULD BE MINOR OR |
| ACCURACY | EQUAL 2.0%. |
| INTERMEDIARY | RELATIVE MEAN DIFFERENCE BETWEEN FIRST |
| ACCURACY | REPETIBILITY MEANS AND SECOND MUST BE LESS |
| | OR EQUAL 2.0% |
| ROBUSTNESS | METHOD WILL BE CONSIDERED ROBUST FOR |
| | SUCH VARIATIONS WHEN THE RESOLUTION |
| | BETWEEN THE CANABIDIOL PEAK AND ANY OTHER |
| | PEAK IS NOT LESS THAN 1.5 AND THE TEST |
| | SOLUTION CONTENT WILL NOT VARY MORE THAN |
| | 2.0% OF THE CONTENT OBTAINED WITHOUT |
| | CHANGE IN ANALYTICAL METHODOLOGY. |

Validation of Analytical Methodology for Related Substances and Degradation Products of the Formulation Object of this Patent.

| EVALUATED | |
|---|---|
| PARAMETER | SPECIFICATION |
| SPECIFICITY | PROOF THAT THE PEAK QUANTIFIED IN THE |
| | CHROMATOGRAM OF THE TEST SOLUTION IS |
| | REALLY THE ANALYTE OF INTEREST (CANABIDIOL) |
| | THROUGH THE COMPARISON OF THE RETENTION |
| | TIME OF THIS PEAK WITH THE RETENTION TIME OF |
| | CANABIDIOL IN THE STANDARD SOLUTION. |
| | PROOF THAT ANY DEGRADATION PRODUCT CO- |
| | ELUATES (RESOLUTION EQUAL TO OR MORE THAN |
| | 1.5) WITH THE CANABIDIOL PEAK, Δ9-THC AND CBN |
| | IN THE TEST SOLUTION THROUGH THE SAMPLES |
| | ANALYSIS SUBMITTED TO FORCED DEGRADATION |
| | IN ACID, BASE, OXIDATIVE, THERMAL, PHOTOLYTIC |
| | MEDIUM, HUMIDITY AND METAL ION AND |
| | POSTERIOR VERIFICATION OF CANABIDIOL PEAK |
| | PURITY IN THESE SOLUTIONS AND POSTERIOR |
| | VERIFICATION OF CANABIDIOL PEAK PURITY, Δ9- |
| | THC AND CBN (IF FORMED) IN THESE SOLUTIONS. |
| | THE RESULTS OF THE PEAK PURITY ARE |
| | EXPRESSED BY COMPARING THE PURITY ANGLE |
| | WITH THE PURITY THRESHOLD WHEN PURITY |
| | ANGLE VALUE IS LESS THAN THRESHOLD PURITY |
| | VALUE, PEAK IS PURE AND, ON CONTRARY, PEAK |
| | IS IMPURE. |
| LINEARITY | CORRELATION COEFFICIENT (R) MUST BE EQUAL |
| | OR GREATER THAN 0.99. |
| | DO NOT PRESENT TREND IN THE DISTRIBUTION |
| | OF RESIDUES. |
| | RELATIVE STANDARD DEVIATION (RSD) BETWEEN |
| | THE 3 SAMPLES OF EACH CONCENTRATION LEVEL |
| | WILL BE ALSO EVALUATED, AND IT SHOULD NOT |
| | EXCEED 11.0% FOR CONCENTRATION <1 μg/mL TO |
| | <10 μg/mL AND SHOULD NOT EXCEED 7.3% FOR |
| | CONCENTRATION ≥10 μg/mL TO 100 μg/mL. |
| RESPONSE | RELATIVE RESPONSE FACTOR WILL BE OBTAINED |
| FACTOR | THROUGH THE RATION BETWEEN THE ANGULAR |
| | COEFFICIENTS FROM IMPURITIES AND ACTIVE, |
| | OBTAINED FROM THE LINEARITY PARAMETER. |
| LIMIT OF | THIS PARAMETER WAS DETERMINED FROM THE |
| QUANTIFICATION | RESULTS OF THE LINEARITY PARAMETER. |
| INTERVAL | THIS PARAMETER IS ESTABLISHED BY THE |
| | CONFIRMATION THAT THE METHOD PROVIDES |
| | PRECISION, ACCURACY AND LINEARITY SUITABLE |
| | WHEN APPLIED TO SAMPLES CONTAINING |
| | SUBSTANCE QUANTITIES WITHIN THE SPECIFIED |
| | INTERVAL. |
| ACCURACY | DIFFERENCE BETWEEN THE ADDED |
| | (THEORETICAL) AND THE RECOVERED |
| | (EXPERIMENTAL) AMOUNTS SHOULD BE BETWEEN |
| | 80% AND 110% FOR IMPURITY CONCENTRATION <1 μg/mL; |
| | BETWEEN 80% AND 110% FOR IMPURITY |
| | CONCENTRATION ≥1 μg/mL AND BETWEEN 99% |
| | AND 107% FOR IMPURITY CONCENTRATION ≥10 μg/mL |
| | TO <100 μg/mL. |
| | RSD BETWEEN 3 SAMPLES OF EACH |
| | CONCENTRATION LEVEL (0.05%, 0.14%, 1.00% AND |
| | 2.00% OF THE NOMINAL CONCENTRATION OF |
| | CANABIDIOL OF 4000,00 μg/mL) AND IT SHOULD NOT |
| | EXCEED 15% FOR IMPURITY CONCENTRATION <1 μg/mL; |
| | 11% FOR IMPURITY CONCENTRATION ≥1 μg/mL |
| | TO <10 μg/mL AND 7.3% FOR IMPURITY |
| | CONCENTRATION ≥10 μg/mL TO <100 μg/mL. |
| REPETIBILITY | REPETIBILITY RESULT EVALUATION WILL BE |
| | PERFORMED BY ASSESSING THE RELATIVE |
| | STANDARD DEVIATION OF EACH LEVEL, THIS |
| | SHOULD NOT EXCEED 15% FOR IMPURITY |
| | CONCENTRATION <1 μg/mL; 11% FOR IMPURITY |
| | CONCENTRATION ≥1 μg/mL TO <10 μg/mL AND 7.3% |
| | FOR IMPURITY CONCENTRATION ≥10 μg/mL TO |
| | <100 μg/mL. |
| INTERMEDIARY | INTERMEDIATE ACCURACY WILL BE ASSESSED |
| ACCURACY | THROUGH THE VARIATION BETWEEN THE MEANS |
| | OF THE CONTENTS OBTAINED AT EACH |
| | CONCENTRATION LEVEL ON THE FIRST AND ON |
| | THE SECOND DAY OF ANALYSIS, WHICH |
| | DIFFERENCE SHOULD BE UPPER 15% FOR |
| | IMPURITY CONCENTRATION <1 μg/mL; 11% FOR |
| | IMPURITY CONCENTRATION ≥1 μg/mL TO <10 μg/mL |
| | AND 7.3% FOR IMPURITY CONCENTRATION ≥10 μg/mL |
| | TO <100 μg/mL. |
| ROBUSTNESS | RESOLUTION BETWEEN THE PEAKS OF |
| | DEGRADATION AND THE CANABIDIOL PEAK ON |
| | EACH CHROMATOGRAM SHOULD NOT BE LESS |
| | THAN 1.5 AND VARIABLES BETWEEN THE CONTENT |
| | OF THE ACCURACY SOLUTION AND THE CONTENT |
| | OBTAINED WITH THE METHOD WITHOUT CHANGES |
| | SHOULD NOT BE UPPER 15.0% WITH RECOVERY OF |
| | 80 TO 110% FOR IMPURITY CONCENTRATION <1 μg/mL, |
| | SHOULD NOT BE UPPER 11.0% WITH |
| | RECOVERY; BETWEEN 80 T0 110% FOR IMPURITY |
| | CONCENTRATION ≥1 μg/mL TO <10 μg/mL, SHOULD |
| | NOT BE UPPER 7.3% WITH RECOVERY BETWEEN 90 |
| | TO 107% FOR IMPURITY CONCENTRATION ≥10 μg/mL |
| | TO <100 μg/mL. |

1. The oral liquid composition characterized by comprising cannabinoids in an oily solvent.

2. A composition according to claim 1, wherein the cannabinoid is selected from the group consisting of endocannabinoids, phytocannabinoids, synthetic cannabinoids and combinations thereof.

3. A composition according to claim 2, wherein the endocannabinoids are selected from the group consisting of anandamide (AEA), 2-arachidonoylglycerol (2-AG), 2-arachidonyl glyceryl ether, N-arachidonoyl dopamine (NADA), virodhamine (OAE), its pharmaceutically acceptable salts or acids, and combinations thereof.

4. A composition according to claim 2 wherein the phytocannabinoids are selected from the group consisting of Δ9-tetrahydrocannabinol (D9-THC), Δ8-tetrahydrocannabinol (D8-THC), tetrahydrocannabinol acid (THC-A), tetrahydrocannabivarin acid (CBD), cannabidiol acid (CBDV), cannabivarine acid (CBDV-A), cannabigerol acid (CBG-A), cannabigerovarine (CBGV), cannabinovarin (CBNV), cannabigerol (CBG), cannabichromene (CBC), cannabicyclol (CBL), cannabivarine (CBV), tetrahydrocannabivarin (THCV), cannabidivarine (CBDV), tetrahydrocannabinol (THC), cannabidiol (CBD), cannabinol (CBN), its pharmaceutically acceptable salts or acids, and combinations thereof.

5. A composition according to claim 2, wherein the synthetic cannabinoids are selected from the group comprising dronabinol, nabilone, rimonabant, its pharmaceutically acceptable salts or acids, and combinations thereof.

6. A composition according to claim 2, wherein the cannabinoid is cannabidiol.

7. A composition according to claim 1, wherein the concentration of the cannabinoid is from 5 to 500 mg/ml.

8. A composition according to claim 1, wherein the oily solvent is selected from the group consisting of grape seed oil, sesame oil, corn oil, soybean oil, olive oil, sunflower oil, canola oil, walnuts oil, linseed oil, avocado oil, peppermint oil, peanuts oil, hydrogenated castor oil, coconut oil, acai oil, andiroba oil, babassu oil, buriti oil, Brazil nut oil, copaiba oil, passion fruit oil, pracaxi oil, patawa oil, triglycerides, primrose oil, safflower oil, almonds oil, borage oil, pomegranate seed oil, sea buckthorn oil, garlic oil, krill oil, cod liver oil, palm oil, fish oil, hydrogenated castor oil, macadamia oil, musk rose oil, cotton oil, and combinations thereof.

9. A composition according to claim 8, wherein the oily solvent is corn oil.

10. A composition according to claim 1, further comprising a co-solvent selected from propylene glycol, glycerol, polyethylene glycol, ethanol, and combinations thereof.

11. The process for preparing an oral liquid composition comprising the steps of:
a. Dissolution of cannabinoid:
i. in the oily solvent; or
ii. in the co-solvent, followed by addition and homogenization to the oily solvent;
b. Addition of the excipients and homogenization.

12. The process according to claim 11, wherein the dissolution step a) occurs under stirring and/or heating.

13. The use of the oral liquid composition according to claim 1, wherein it is for the preparation of a medicament to be used in the treatment of refractory epilepsy, epilepsy, Parkinson's disease, schizophrenia, sleep disorders, post-traumatic disorder, anxiety, chronic pain relief and autism.

14. Validated analytical methodology for assuring identity, quality and purity of Active Pharmaceutical Ingredient and the composition according to claim 10, characterized in that it is consisted of the following steps: (i).
